(19) United States
(12) Patent Application Publication
(10) Pub. No.: US 2026/0165269 A1
(43) Pub. Date: Jun. 18, 2026

(54) PRIMER SET AND KIT FOR IDENTIFYING ASTRAGALUS MEMBRANACEUS (FISCH.) BGE. VAR. MONGHOLICUS (BGE.) HSIAO AND IDENTIFICATION METHOD FOR ASTRAGALUS MEMBRANACEUS (FISCH.) BGE. VAR. MONGHOLICUS (BGE.) HSIAO

(71) Applicants: Shanxi Agricultural University Economic Crop Research Institute, Taiyuan (CN); Shanxi Agricultural University Agricultural Gene Resources Research Center, Taiyuan (CN)

(72) Inventors: Hongling TIAN, Taiyuan (CN); Lijun ZHANG, Taiyuan (CN); Yaoqin WANG, Taiyuan (CN); Shuhong GUO, Taiyuan (CN)

(73) Assignees: Shanxi Agricultural University Economic Crop Research Institute, Taiyuan (CN); Shanxi Agricultural University Agricultural Gene Resources Research Center, Taiyuan (CN)

(21) Appl. No.: 19/422,488

(22) Filed: Dec. 17, 2025

(30) Foreign Application Priority Data
Dec. 17, 2024 (CN) 202411866677.3

(51) Int. Cl. A01H 1/04; C12Q 1/6806; C12Q 1/686; C12Q 1/6895

(52) U.S. Cl. CPC A01H 1/045; C12Q 1/6806; C12Q 1/686; C12Q 1/6895; C12Q 2600/13

(57) ABSTRACT

A primer set for identifying Astragalus membranaceus (Fisch.) Bge. var. mongholicus (Bge.) Hsiao and an identification method for Astragalus membranaceus (Fisch.) Bge. var. mongholicus (Bge.) Hsiao are provided. The primer set includes a primer 18678_1F and a primer 18678_1R, wherein the 18678_1F has a nucleotide sequence set forth in SEQ ID NO: 1, and the 18678_1R has a nucleotide sequence set forth in SEQ ID NO: 2. The primer set is designed for SSR molecular markers. The SSR molecular markers have the advantages of uniform distribution, rich polymorphism, good amplification repeatability, and clear banding in the Astragalus genome. The SSR molecular markers are suitable for detection by a common denaturing polyacrylamide gel electrophoresis detection platform and a capillary fluorescence detection platform, and can be further applied to research fields such as Radix astragali genetic diversity analysis, variety identification, DNA fingerprint map construction, and molecular marker-assisted breeding.

### CROSS REFERENCE TO THE RELATED APPLICATIONS

This application is based upon and claims priority to Chinese Patent Application No. 202411866677.3, filed on Dec. 17, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC254_SequenceListing.xml, created on Nov. 27, 2025, and is 170,353 bytes in size.

### TECHNICAL FIELD

The present invention belongs to the technical field of molecular markers, and particularly relates to a primer set and a kit for identifying Astragalus membranaceus (Fisch.) Bge. var. mongholicus (Bge.) Hsiao and an identification method for Astragalus membranaceus (Fisch.) Bge. var. mongholicus (Bge.) Hsiao.

### BACKGROUND

Radix astragali, originally called “Huangqi”, tastes sweet, is slightly warm and not dry, has been listed as a top-grade medicine in “Divine Farmer's Classic of Materia Medica”, and is a commonly used tonic medicine in clinical practice. Radix astragali has the effects of invigorating qi, invigorating vital function, consolidating superficial resistance, suppressing sweating, promoting diuresis, eliminating the swelling, promoting secretion, enriching the blood, removing stagnation and arthralgia, reinforcing qi and blood, promoting pus discharge, promoting wound healing and promoting granulation. Modern medical research shows that Radix astragali can enhance body immunity, resist aging and viruses and strengthen heart function.

The quality Radix astragali includes Astragalus membranaceus (Fisch.) Bge. var. mongholicus (Bge.) Hsiao and Astragalus membranaceus (Fisch.) Bge. Both Astragalus membranaceus (Fisch.) Bge. var. mongholicus (Bge.) Hsiao and Astragalus membranaceus (Fisch.) Bge. are diploid plants. Traditional Chinese medicine identification methods, such as macroscopic identification and microscopic identification, have inherent defects such as low accuracy, strong subjective influence, and low versatility, and can no longer meet the requirements of the modernized traditional Chinese medicine market which is developed vigorously. How to use a simple, accurate and rapid method to distinguish authenticity and distinguish quality has become a difficult problem facing many Chinese medicine researchers.

Molecular markers are a fourth generation of genetic markers after morphological markers, cytological markers, and protein markers. The molecular markers have many advantages, such as a large number of markers, markers not being affected by the environment, simple operation, and low development costs.

The molecular markers such as SSR, AFLP, and RAPD can all be used to analyze the genetic diversity of Radix astragali, wherein the SSR molecular markers have the following advantages over other markers: (1) SSR is abundant and widely distributed throughout the genome; (2) the experimental repeatability is good and the results are reliable; (3) SSR has more allelic variation; (4) SSR has low requirements on DNA quality, requires less DNA, and does not require the use of isotopes; (5) SSR is a co-dominant marker and can identify heterozygous and homozygous genotypes. Therefore, the development of SSR molecular markers can present as many genetic differences of these samples as possible at the molecular level, thereby eliminating the false and the inferior and retaining the true and the superior. In addition, performing genetic mapping, gene positioning, genetic diversity analysis, and breeding of improved varieties based on molecular markers is helpful to further promote the development of Radix astragali towards high quality, standardization, and industrialization.

In the prior art, Chinese Patent No. CN109735643A (published on May 10, 2019) discloses a method for identifying or assisting in identifying Radix astragali based on single nucleotide polymorphism markers, which includes the following steps: 1) extracting DNA of to-be-detected Radix astragali as a template; and 2) through sequencing, if the 476^{th} site of an ITS sequence is T base, determining the to-be-detected Radix astragali as Astragalus membranaceus (Fisch.) Bge., and if the 476th site of an ITS sequence is C base, determining the to-be-detected Radix astragali as Astragalus membranaceus (Fisch.) Bge. var. mongholicus (Bge.) Hsiao. This technology mainly uses single nucleotide mutations in ITS sequences to identify Radix astragali, which requires sequencing.

### SUMMARY

In view of this, the present invention aims to provide a primer set and a kit for identifying Astragalus membranaceus (Fisch.) Bge. var. mongholicus (Bge.) Hsiao and an identification method for Astragalus membranaceus (Fisch.) Bge. var. mongholicus (Bge.) Hsiao. The primer set provided by the present invention is designed for SSR molecular markers. The SSR molecular markers have the advantages of uniform distribution, rich polymorphism, good amplification repeatability, and clear banding in the Astragalus genome. The SSR molecular markers are suitable for detection by a common denaturing polyacrylamide gel electrophoresis detection platform and a capillary fluorescence detection platform, and can be further applied to research fields such as Radix astragali genetic diversity analysis, variety identification, DNA fingerprint map construction, and molecular marker-assisted breeding.

The present invention provides a primer set for identifying Astragalus membranaceus (Fisch.) Bge. var. mongholicus (Bge.) Hsiao, which includes a primer 18678_1F and a primer 18678_1R, wherein the 18678_1F has a nucleotide sequence set forth in SEQ ID NO: 1, and the 18678_1R has a nucleotide sequence set forth in SEQ ID NO: 2.

The present invention provides a kit for identifying Astragalus membranaceus (Fisch.) Bge. var. mongholicus (Bge.) Hsiao, which includes the primer set and a detection reagent.

Preferably, the detection reagent is a polymerase chain reaction (PCR) amplification reagent.

The present invention provides a method for identifying Astragalus membranaceus (Fisch.) Bge. var. mongholicus (Bge.) Hsiao by using the primer set, which includes the following steps:
1) extracting genomic DNA from a to-be-detected sample;
2) performing PCR amplification by using the genomic DNA obtained in the step 1) as a template and using the primer set to obtain an amplification product; and
3) detecting the amplification product obtained in the step 2), and if the amplification product has a single 144 bp sequence, determining that the to-be-detected sample is Astragalus membranaceus (Fisch.) Bge. var. mongholicus (Bge.) Hsiao.

Preferably, a material for extracting genomic DNA of the to-be-detected sample in step 1) is a seedling leaf.

Preferably, the template in step 2) has a concentration of 10-20 ng/μL.

Preferably, the primer set in step 2) has a concentration of 8-12 μM.

Preferably, the PCR amplification system in step 2), with a total volume of 20 μL, includes the following components: 1 μL of template, 0.5 μL of the primer 18678_1F, 0.5 μL of the primer 18678_1R, 10 μL of 2×MIX, and 8 μL of ddH₂O;
a program of the PCR amplification in the step 2) is as follows: pre-denaturation at 95° C. for 5 min; 15 cycles of denaturation at 95° C. for 30 s, annealing at 60° C. for 30 s and extension at 72° C. for 30 s; 20 cycles of pre-denaturation at 95° C. for 30 s, annealing at 50° C. for 30 s and extension at 72° C. for 30 s; finally extension at 72° C. for 7 min.

Preferably, a method for detecting the amplification product includes polyacrylamide gel electrophoresis or fluorescent labeling capillary electrophoresis.

The present invention further provides use of the primer set or the kit in Radix astragali-assisted breeding.

Compared with the prior art, the present invention has the following beneficial effects: the primer set for identifying Astragalus membranaceus (Fisch.) Bge. var. mongholicus (Bge.) Hsiao is an SSR primer set, and the present invention performs DNA extraction on Radix Astragali germplasm resources, constructs a genomic library by mixing samples, performs sequencing and searches SSR sites; designs primers for a searched SSR sequence, and detects primer polymorphism to obtain a primer set provided by the present invention. The primer set has good amplification repeatability and clear band pattern, and can be further applied to research fields such as Radix astragali genetic diversity analysis, variety identification, DNA fingerprint map construction, and molecular marker-assisted breeding.

### BRIEF DESCRIPTION OF THE DRAWINGS

FIG. 1 is a first electrophoretogram (104923-19262) of an amplification product obtained by PCR amplification using primers;

FIG. 2 is a second electrophoretogram (20195-26222) of an amplification product obtained by PCR amplification using primers;

FIG. 3 is a third electrophoretogram (26818-47749) of an amplification product obtained by PCR amplification using primers;

FIG. 4 is a fourth electrophoretogram (483-900) of an amplification product obtained by PCR amplification using primers; and

FIG. 5 is a fifth electrophoretogram (9129-1859) of an amplification product obtained by PCR amplification using primers.

### DETAILED DESCRIPTION OF THE EMBODIMENTS

The present invention provides a primer set for identifying Astragalus membranaceus (Fisch.) Bge. var. mongholicus (Bge.) Hsiao, which includes a primer 18678_1F and a primer 18678_1R, wherein the 18678_1F has a nucleotide sequence set forth in SEQ ID NO: 1, and the 18678_1R has a nucleotide sequence set forth in SEQ ID NO: 2, as shown in Table 1.

TABLE 1
Primer set sequence

| | | | Amplifi- |
|---|---|---|---|
| | | | cation |
| Primer | | | product |
| length | Primer | Sequence | length |
| (bp) | name | (3′-5′) | (bp) |
| 20 | 18678_ | AAACCCAAACGAAGCACAAC | 144 |
| | 1F | (SEQ ID NO: 1) | |
| 20 | 18678_ | GAAAACAAAATCCGTCCCAA | |
| | 1R | (SEQ ID NO: 2) | |

The present invention further provides a kit for identifying Astragalus membranaceus (Fisch.) Bge. var. mongholicus (Bge.) Hsiao, which includes the primer set and a detection reagent.

In the present invention, the detection reagent is preferably a PCR amplification reagent. The present invention has no special limitation on the source of the PCR amplification reagent, and conventional commercial products in the art can be used.

The present invention provides a method for identifying Astragalus membranaceus (Fisch.) Bge. var. mongholicus (Bge.) Hsiao by using the primer set, which includes the following steps:
1) extracting genomic DNA from a to-be-detected sample;
2) performing PCR amplification by using the genomic DNA obtained in the step 1) as a template and using the primer set to obtain an amplification product; and
3) detecting the amplification product obtained in the step 2), and if the amplification product has a single 144 bp sequence, determining that the to-be-detected sample is Astragalus membranaceus (Fisch.) Bge. var. mongholicus (Bge.) Hsiao.

In the present invention, the genomic DNA of the to-be-detected sample is first extracted. The material for extracting the genomic DNA of the to-be-detected sample is preferably a seedling leaf. The present invention has no special limitation on the method for extracting the genomic DNA, and the plant genomic DNA extraction method known in the art can be used.

After the genomic DNA is obtained, the obtained genomic DNA is used as a template, and the primer set is used for PCR amplification to obtain an amplification product. In the present invention, the concentration of the template is preferably 10-20 ng/μL, more preferably 12-18 ng/μL, and most preferably 15 ng/μL, and the concentration of the primer set is preferably 8-12 μM, more preferably 9-11 μM, and even more preferably 10 μM.

In the present invention, the PCR amplification system, with a total volume of 20 μL, includes the following components: 1 μL of template, 0.5 μL of the primer 18678_1F, 0.5 μL of the primer 18678_1R, 10 μL of 2×MIX, and 8 μL of ddH₂O. The PCR amplification program is preferably pre-denaturation at 95° C. for 5 min; 15 cycles of denaturation at 95° C. for 30 s, annealing at 60° C. for 30 s and extension at 72° C. for 30 s; 20 cycles of pre-denaturation at 95° C. for 30 s, annealing at 50° C. for 30 s and extension at 72° C. for 30 s; finally extension at 72° C. for 7 min.

After the amplification product is obtained, the obtained amplification product is detected, and if the amplification product has a single 144 bp sequence, the to-be-detected sample is Astragalus membranaceus (Fisch.) Bge. var. mongholicus (Bge.) Hsiao. In the present invention, a method for detecting the amplification product includes polyacrylamide gel electrophoresis or fluorescent labeling capillary electrophoresis.

The present invention further provides use of the primer set or the kit in Radix astragali-assisted breeding.

The technical solutions provided by the present invention will be described in detail below with reference to examples, which, however, should not be construed as limiting the scope of the present invention.

### Example 1

Obtaining a genome sequence: DNA was extracted from 9 representative selected Radix astragali germplasm resources (as shown in Table 2), and the genomic library was constructed by mixing samples. The core of SSR and the conserved sequence regions at two ends were enriched by magnetic beads for sequencing. This part of the work was entrusted to Nanjing Personal Biotechnology Co., Ltd., and the project name was SSR molecular marker development (YFnj20224223).

TABLE 2
Representative Radix Astragali germplasm resources

| No. | Name | Source |
|---|---|---|
| A1 | 1 Astragalus membranaceus (Fisch.) Bge. var. | Guaner Township, Hunyuan |
| | mongholicus (Bge.) Hsiao green covered with hair | |
| A2 | 2 Astragalus membranaceus (Fisch.) Bge. var. | Peicun Township, Hunyuan |
| | mongholicus (Bge.) Hsiao green hairless | |
| A3 | 3 Astragalus membranaceus (Fisch.) Bge. var. | Peicun Township, Hunyuan |
| | mongholicus (Bge.) Hsiao green hairless red tip | |
| A4 | 4 Astragalus membranaceus (Fisch.) Bge. var. | Shagetuo Township, Hunyuan |
| | mongholicus (Bge.) Hsiao red hairless | |
| A5 | 5 Astragalus membranaceus (Fisch.) Bge. var. | Qingciyao Township, Hunyuan |
| | mongholicus (Bge.) Hsiao red covered with hair | |
| A6 | 6 Astragalus membranaceus (Fisch.) Bge. red | Heyeping, Wuzhai County |
| | covered with hair | |
| A7 | 7 Astragalus membranaceus (Fisch.) Bge. green | Hezheng County, Gansu Province |
| | covered with hair | |
| A8 | 8 Astragalus membranaceus (Fisch.) Bge. green | Hezheng County, Gansu Province |
| | hairless | |
| A9 | 9 Astragalus membranaceus (Fisch.) Bge. red | Hezheng County, Gansu Province |
| | hairless | |

(2) Searching for genomic sequences containing SSR: SSR sequences were searched in the sequenced sequences and the search results of SSR were counted.

(3) Designing SSR primer: the SSRs were then clustered and polymorphisms were evaluated. Primers for polymorphic SSRs were designed. The designed primers are shown in Table 3.

TABLE 3
List of designed SSR primers

| | | | PRODUCT1 |
|---|---|---|---|
| | Cluster | | size |
| | ID | FORWARD PRIMER1 (5′-3′) | (bp) |
| 1 | 104923_1F | AAAATGGAAGAGTGTGGAAGAAA(SEQ ID NO: 3) | 90 |
| | 104923_1R | TCTCCTCTCCTCACATTCGC(SEQ ID NO: 4) | |
| 2 | 10779_1F | ACATTATTTCCCATGCCCAA(SEQ ID NO: 5) | 184 |
| | 10779_1R | CAAAATGGAAGAGTGTGGAAGA(SEQ ID NO: 6) | |
| 3 | 11109_1F | CTTGATGTTCTTGGTTGCTCA(SEQ ID NO: 7) | 113 |
| | 11109_1R | CACACATAGGTCGCATCACC(SEQ ID NO: 8) | |
| 4 | 11156_1F | ATGGTTGAGCTCCAAAATCG(SEQ ID NO: 9) | 287 |
| | 11156_1R | ACCCTCAGCTGAAAAACACC(SEQ ID NO: 10) | |
| 5 | 11230_2F | AACTTAGGGGTGATGGGAGG(SEQ ID NO: 11) | 254 |
| | 11230_2R | ATCGTTCACCGGACGATAAG(SEQ ID NO: 12) | |
| 6 | 1128_1F | AAACCAGCTGGACCAAACAC(SEQ ID NO: 13) | 228 |
| | 1128_1R | AAAGGGTTGCTGGGAGTTTT(SEQ ID NO: 14) | |
| 7 | 11337_1F | AAGGCAAAGCACACAAAGGT(SEQ ID NO: 15) | 209 |
| | 11337_1R | GGTCCCCTCGTTCTTTTCTT(SEQ ID NO: 16) | |
| 8 | 11852_1F | AACGCTTTCTTTTATTTCTCGC(SEQ ID NO: 17) | 173 |
| | 11852_1R | GCGGAATTGTGAATGACAAGA(SEQ ID NO: 18) | |
| 9 | 12023_1F | ACCACAGAACCAGAAATGCC(SEQ ID NO: 19) | 261 |
| | 12023_1R | CCAGCAACCAAAAACTCAGA(SEQ ID NO: 20) | |
| 10 | 13775_1F | AGCGGACCTGTCCAATACAG(SEQ ID NO: 21) | 176 |
| | 13775_1R | TTTTTGAATCACCCATGAACC(SEQ ID NO: 22) | |
| 11 | 13784_1F | CAGGCTCACGACGTTAAACA(SEQ ID NO: 23) | 231 |
| | 13784_1R | AAACTCTCCTCCCCCACACT(SEQ ID NO: 24) | |
| 12 | 13961_1F | ACTCCTTGATTTTCTCTTCCACT(SEQ ID NO: 25) | 207 |
| | 13961_1R | TTTAGGCTTCTTTTGCGTGAA(SEQ ID NO: 26) | |
| 13 | 14132_1F | AACGCTTTCTTTTATTTCTCGC(SEQ ID NO: 17) | 217 |
| | 14132_1R | CCACAGGGAGAGCCTAAGTG(SEQ ID NO: 27) | |
| 14 | 15250_1F | AAAATTATTTCCAAAAATACAGTGAGA(SEQ ID NO: 28) | 119 |
| | 15250_1R | TTCATTCTTAAAAGCGTCTGTGTC(SEQ ID NO: 29) | |
| 15 | 15349_1F | CCAGATCTGTTCAACAACATTCA(SEQ ID NO: 30) | 100 |
| | 15349_1R | GGCAAATTGAGAAACCCAGA(SEQ ID NO: 31) | |
| 16 | 17010_1F | AAACGATTGAAACGAAACAAA(SEQ ID NO: 32) | 85 |
| | 17010_1R | CCGAAACTGATCGCTCTCTC(SEQ ID NO: 33) | |
| 17 | 17094_1F | CACCCGTGAGCGTCTTATTT(SEQ ID NO: 34) | 236 |
| | 17094_1R | CCACTATCTTCTCTCTCACAATTCC(SEQ ID NO: 35) | |
| 18 | 17765_1F | ACCCAGGTTGGTAAGCTCCT(SEQ ID NO: 36) | 282 |
| | 17765_1R | AAGACGCATTCAATCGGAAC(SEQ ID NO: 37) | |
| 19 | 17817_1F | CATTCAATCCGCCTCAAGAT(SEQ ID NO: 38) | 268 |
| | 17817_1R | GGAGAAAGAAGAAAAGAAAAGAAGG(SEQ ID NO: 39) | |
| 20 | 18678_1F | AAACCCAAACGAAGCACAAC(SEQ ID NO: 1) | 144 |
| | 18678_1R | GAAAACAAAATCCGTCCCAA(SEQ ID NO: 2) | |
| 21 | 19118_1F | ACAAATTTGGCCGCTACAAC(SEQ ID NO: 40) | 149 |
| | 19118_1R | AAGACGAGTTTCTGTAAAATTGGG(SEQ ID NO: 41) | |
| 22 | 19237_1F | AACTAGCAACCCATAGTGATAAAAA(SEQ ID NO: 42) | 249 |
| | 19237_1R | GGAAGAGGCTCCTCTGGTCT(SEQ ID NO: 43) | |
| 23 | 19258_1F | CCCTATTTTGACATTAAAGTTGTGAA(SEQ ID NO: 44) | 187 |
| | 19258_1R | GAAGAAGGCAATGCACACAA(SEQ ID NO: 45) | |
| 24 | 19262_1F | CACACACGCACACACTTGAG(SEQ ID NO: 46) | 199 |
| | 19262_1R | TGGAGGGTGTCGAAGTTTCT(SEQ ID NO: 47) | |
| 25 | 20195_1F | AGAGCACGCGTTTTCAGTTT(SEQ ID NO: 48) | 105 |
| | 20195_1R | CACACACGCACACACTTGAG(SEQ ID NO: 46) | |
| 26 | 20486_1F | AAATCCCTCGCTAACCAAAAA(SEQ ID NO: 49) | 202 |
| | 20486_1R | GTGAAGTTCGCGAGTGAGG(SEQ ID NO: 50) | |
| 27 | 20590_1F | AATCGCATCTCACTTTTGGG(SEQ ID NO: 51) | 159 |
| | 20590_1R | CGAAGTAGATGCAGAGCGAG(SEQ ID NO: 52) | |
| 28 | 20703_1F | CGTAGAGGACCAAACCGAAA(SEQ ID NO: 53) | 215 |
| | 20703_1R | AACAGAAAAACGAAAGAGCGA(SEQ ID NO: 54) | |
| 29 | 20771_1F | AAAGAAAGAAAGGCATCGCA(SEQ ID NO: 55) | 194 |
| | 20771_1R | CCTCTACGAAAGCACCGAAG(SEQ ID NO: 56) | |
| 30 | 21073_1F | CCATCTGCCTCACCATTCTT(SEQ ID NO: 57) | 251 |
| | 21073_1R | GAAGGAAGAGGAAGTGGAGAAA(SEQ ID NO: 58) | |
| 31 | 21176_1F | CACATGTGTGAGGACGAGAGA(SEQ ID NO: 59) | 263 |
| | 21176_1R | ATGCAAAACAAGGGTTGGAG(SEQ ID NO: 60) | |
| 32 | 21582_1F | ATGTTGCCGGTTTGATTCAT(SEQ ID NO: 61) | 287 |
| | 21582_1R | CAACCAACAACCAACAACCA(SEQ ID NO: 62) | |
| 33 | 2184_1F | AGACAGTGCGAAGGAGGAAA(SEQ ID NO: 63) | 188 |
| | 2184_1R | CACCCGTCATGTTTGATTTG(SEQ ID NO: 64) | |
| 34 | 22148_1F | AAAGAAACGAGTGGTGGTGG(SEQ ID NO: 65) | 111 |
| | 22148_1R | CTCCTCACACTCTTCTCCCA(SEQ ID NO: 66) | |
| 35 | 22821_1F | TCATTGTTAAAAGCGTCTGTGTC(SEQ ID NO: 67) | 227 |
| | 22821_1R | TTCATCAAGACTGTTATAATTGGTAAA(SEQ ID NO: 68) | |
| 36 | 23049_1F | AAGGGAGAGAGAGGAGGTCG(SEQ ID NO: 69) | 148 |
| | 23049_1R | GTGGAAAGAGGTTGGAGGGT(SEQ ID NO: 70) | |
| 37 | 23079_1F | ATGAACCCCACCCTTACCTT(SEQ ID NO: 71) | 150 |
| | 23079_1R | AGAAGGCGTGAATCCACAAG(SEQ ID NO: 72) | |
| 38 | 2318_1F | ACAGACCCCATTCACCAAAA(SEQ ID NO: 73) | 187 |
| | 2318_1R | TGTTTGGTTTGGAAATCGCT(SEQ ID NO: 74) | |
| 39 | 23186_1F | AGGAAAACCGAAGGGAGAAA(SEQ ID NO: 75) | 103 |
| | 23186_1R | CATGCAATGACAATGGAGATG(SEQ ID NO: 76) | |
| 40 | 23194_1F | CCGCTTTCTTTTATTTCACG(SEQ ID NO: 77) | 110 |
| | 23194_1R | GAAAGCGGTAAATTGCATGA(SEQ ID NO: 78) | |
| 41 | 23547_1F | AAATTTTGACTAAAAAGGGAAATACA(SEQ ID NO: 79) | 248 |
| | 23547_1R | TTTCATTGTTAAAAGTGTGTGTGTCT(SEQ ID NO: 80) | |
| 42 | 24189_1F | GGGGTAATTGTGTCACATCCA(SEQ ID NO: 81) | 287 |
| | 24189_1R | TTAGGCTTCTTTTGCGTGAA(SEQ ID NO: 82) | |
| 43 | 24276_1F | ATTCCCATTATTTCCCAGGC(SEQ ID NO: 83) | 191 |
| | 24276_1R | CAAAATGGAAGAGTGTGGAAGA(SEQ ID NO: 6) | |
| 44 | 24844_1F | CGCCCGATCTGTAACGTACT(SEQ ID NO: 84) | 263 |
| | 24844_1R | GGAGTGTTAATACTGCAACGCA(SEQ ID NO: 85) | |
| 45 | 25144_1F | ATCAGTAGCGGCAAGCAAAC(SEQ ID NO: 86) | 145 |
| | 25144_1R | AAGTCAAAACCTGCTGGGTG(SEQ ID NO: 87) | |
| 46 | 25607_1F | AGTGATGCAATGTGCAGAGC(SEQ ID NO: 88) | 232 |
| | 25607_1R | CGTTCATCGTCATCGTGATT(SEQ ID NO: 89) | |
| 47 | 2593_1F | AAAAATCCAACGGTCTCGTG(SEQ ID NO: 90) | 243 |
| | 2593_1R | AAAACCAAACACCCAACCAC(SEQ ID NO: 91) | |
| 48 | 26222_1F | GAAGGGAGAGAGAGAAAATTCG(SEQ ID NO: 92) | 177 |
| | 26222_1R | GTGCACATTCACTTCCATGC(SEQ ID NO: 93) | |
| 49 | 26818_1F | CTTGGTGTGATTGGTGTGCT(SEQ ID NO: 94) | 217 |
| | 26818_1R | GACCATGACAATGCATCAGG(SEQ ID NO: 95) | |
| 50 | 2778_1F | AAAGAGCACGGAATGGAATG(SEQ ID NO: 96) | 175 |
| | 2778_1R | TGCTCCAGCTGAGAAGAAGA(SEQ ID NO:97) | |
| 51 | 28406_1F | ACCACAACACTGGACCTCAA(SEQ ID NO: 98) | 98 |
| | 28406_1R | TCACATTTCACAACAACACTCAA(SEQ ID NO: 99) | |
| 52 | 2915_1F | CCGCTTTCTTTTATTTCTCGC(SEQ ID NO: 100) | 152 |
| | 2915_1R | GAAAGCGGTAAATTGCATGA(SEQ ID NO: 78) | |
| 53 | 2990_1F | CGGTGACAAAAAGTTGGTAAA(SEQ ID NO: 101) | 150 |
| | 2990_1R | TTGGGGATACGCTTCAAGTC(SEQ ID NO: 102) | |
| 54 | 3040_1F | AAAATCTTCTCTCTCACAATTCCA(SEQ ID NO: 103) | 183 |
| | 3040_1R | CGTGAGTGCGTTTTTAAGGC(SEQ ID NO: 104) | |
| 55 | 31895_1F | AGGGGTAATTGTGTCGCATC(SEQ ID NO: 105) | 293 |
| | 31895_1R | TTTAGGCTTCTTTTGCGTGAA(SEQ ID NO: 26) | |
| 56 | 332_1F | ATACGCGCTGATTAGGATGC(SEQ ID NO: 105) | 270 |
| | 332_1R | TCATGACCATATTGAGAGATCAGA(SEQ ID NO: 107) | |
| 57 | 33651_1F | AAACCTTGTCGCAGTATGGG(SEQ ID NO: 108) | 225 |
| | 33651_1R | GACGACAACGAGAACGACAA(SEQ ID NO: 109) | |
| 58 | 33656_1F | GATTCTGGGATGTGTTTGGC(SEQ ID NO: 110) | 257 |
| | 33656_1R | GGTCAGAGAGAAAAGGGATGG(SEQ ID NO: 111) | |
| 59 | 34315_1F | AAATTGTTATGTCTGCTAATGTGGA(SEQ ID NO: 112) | 89 |
| | 34315_1R | AGACCAATAGGGCACACAGC(SEQ ID NO: 113) | |
| 60 | 3479_1F | ACTCCTTGATTTTCTCTTCCACT(SEQ ID NO: 25) | 207 |
| | 3479_1R | TGCGTGAAAAATAAAGAAAGATATG(SEQ ID NO: 114) | |
| 61 | 35576_1F | CAACTGGGTTGATTTTGATTG(SEQ ID NO: 115) | 190 |
| | 35576_1R | CCTGCTGCAACAGTGTGTTC(SEQ ID NO: 116) | |
| 62 | 36408_1F | AGAACTCAGCCTCACCATGC(SEQ ID NO: 117) | 229 |
| | 36408_1R | ACGACTAACCCATGCCAATC(SEQ ID NO: 118) | |
| 63 | 39712_1F | AAGGGGAATGGGAAAGATTG(SEQ ID NO: 119) | 215 |
| | 39712_1R | CACTATGGTCATTTCGCCCT(SEQ ID NO: 120) | |
| 64 | 39858_1F | CTTTGCGTGAGTGCGTTTT(SEQ ID NO: 121) | 298 |
| | 39858_1R | GGGTATTTGTGTCACATCCCTT(SEQ ID NO: 122) | |
| 65 | 40857_1F | ACTCTTGGAGAGCCACCTGA(SEQ ID NO: 123) | 280 |
| | 40857_1R | AATTCTCCTCCCCCACACTT(SEQ ID NO: 124) | |
| 66 | 41786_1F | ATGATTTTGCAATTTACCGC(SEQ ID NO: 125) | 198 |
| | 41786_1R | AAGCGAGAAATAAAAGAAAGCG(SEQ ID NO: 126) | |
| 67 | 43989_1F | CAAAATGGATGAATAATGCGAA(SEQ ID NO: 127) | 195 |
| | 43989_1R | ATGGCGTTGAATTTGTAGGC(SEQ ID NO: 128) | |
| 68 | 45903_1F | ACGCAATTTACAATTCTGTCATT(SEQ ID NO: 129) | 193 |
| | 45903_1R | CCCAATAACCCGAGACTTTG(SEQ ID NO: 130) | |
| 69 | 4673_1F | ACCCGTGGGCTTCTTATTTT(SEQ ID NO: 131) | 198 |
| | 4673_1R | ACTCCTTGACTTTCTCTTCCACT(SEQ ID NO: 132) | |
| 70 | 475_1F | CCCCCGGTTAACTCTTCAAT(SEQ ID NO: 133) | 296 |
| | 475_1R | CGAGATTCAAGAAGAAGGGAA(SEQ ID NO: 134) | |
| 71 | 4754_1F | CCCAAGACTTTGTATCTACTCACTCA(SEQ ID NO: 135) | 112 |
| | 4754_1R | CCGCTTTCTTTTATTTCTCGC(SEQ ID NO: 100) | |
| 72 | 47749_1F | CATCTTCCTCTTCTTCACTCTCAG(SEQ ID NO: 136) | 194 |
| | 47749_1R | AGATGATGATGCACAACCGA(SEQ ID NO: 137) | |
| 73 | 483_1F | AACACATCATGGCTCCAACA(SEQ ID NO: 138) | 212 |
| | 483_1R | GCCGTGTGTGGATGTTGATA(SEQ ID NO: 139) | |
| 74 | 49546_1F | CAATTTTATCTTACGTTCATCTGTCA(SEQ ID NO: 140) | 220 |
| | 49546_1R | AAAGGAAGAGCGTGCTTCAA(SEQ ID NO: 141) | |
| 75 | 5150_1F | AAGGAAAGGGCTGGTGAAAT(SEQ ID NO: 142) | 252 |
| | 5150_1R | GATCAAAATATGAAATGTGATGAGA(SEQ ID NO: 143) | |
| 76 | 5301_1F | CATTGTTAAAAGCGCCTGTG(SEQ ID NO: 144) | 240 |
| | 5301_1R | AATTTTGACTAAAAAGGGAAATACAT(SEQ ID NO: 145) | |
| 77 | 53411_1F | AACTCAAGAGACGACCACGC(SEQ ID NO: 146) | 101 |
| | 53411_1R | GATTGTCGGGCAGTGTATCC(SEQ ID NO: 147) | |
| 78 | 53615_1F | AAGGAGGAAATTCCATTCTGC(SEQ ID NO: 148) | 118 |
| | 53615_1R | GATGGGCATCCATCGTTTAC(SEQ ID NO: 149) | |
| 79 | 53709_1F | CAGGAGGCAACCTGTGTTTT(SEQ ID NO: 150) | 172 |
| | 53709_1R | TGATCAAATAGAAACAAAAGGAAA(SEQ ID NO: 151) | |
| 80 | 55206_1F | ACAATCTCAGCGATGCAATG(SEQ ID NO: 152) | 136 |
| | 55206_1R | ACTCACACACACGCACACA(SEQ ID NO: 153) | |
| 81 | 5697_1F | ACCTGTGGGAAACAGGTGAG(SEQ ID NO: 154) | 106 |
| | 5697_1R | GATTGACGGCCTTGTGTTTT(SEQ ID NO: 155) | |
| 82 | 6203_1F | AAAGGAGAACATGCAGGGTG(SEQ ID NO: 156) | 149 |
| | 6203_1R | CATTGATGAAACAGAATTTTCAAGA(SEQ ID NO: 157) | |
| 83 | 63960_1F | ACCACCATCTCTGATGAGCC(SEQ ID NO: 158) | 107 |
| | 63960_1R | TTGGTGTTCTTGAGGAGAGAGA(SEQ ID NO: 159) | |
| 84 | 67487_1F | AACGAAATGCAAAAGGGAGA(SEQ ID NO: 160) | 204 |
| | 67487_1R | CGAGAAGCAGAGAAGGAAAGA(SEQ ID NO: 161) | |
| 85 | 6771_1F | ATCAGCGCCTATTTCAACCT(SEQ ID NO: 162) | 235 |
| | 6771_1R | CCGACCTTCGCAACAGTAAT(SEQ ID NO: 163) | |
| 86 | 7356_1F | CCATGCCCAAAAATTCAAAA(SEQ ID NO: 164) | 226 |
| | 7356_1R | CCAAAGAGGGAAATTTGGGT(SEQ ID NO: 165) | |
| 87 | 73932_1F | AATCGCAAAATTGGTGCTTT(SEQ ID NO: 166) | 176 |
| | 73932_1R | CACCAAATTAAATTCACCAAACC(SEQ ID NO: 167) | |
| 88 | 7457_1F | AAAGAAAAGAAAGAAAGGAAGCG(SEQ ID NO: 168) | 140 |
| | 7457_1R | TCTCTCCCTTTCACCCAGTC(SEQ ID NO: 169) | |
| 89 | 7557_1F | ACGTTCATCTGTCACGTTTTT(SEQ ID NO: 170) | 134 |
| | 7557_1R | TTTAGGCTTCTTTTGCGTGAA(SEQ ID NO: 26) | |
| 90 | 7678_1F | ATTTTGCGTGAGTGCGTTTT(SEQ ID NO: 171) | 224 |
| | 7678_1R | TTGCTCCTTGATTTTCTCTTCC(SEQ ID NO: 172) | |
| 91 | 78064_2F | ACCTTTGTGTGCATTGCCTT(SEQ ID NO: 173) | 164 |
| | 78064_2R | TCGAAAATGAAAGTGATGATGAA(SEQ ID NO: 174) | |
| 92 | 78968_1F | CCCTCTTCTGCCATTGGTAA(SEQ ID NO: 175) | 206 |
| | 78968_1R | AGGACTTACCCTAAACCCGC(SEQ ID NO: 176) | |
| 93 | 7982_1F | AAGGGGGAAGTGTCCTCAAT(SEQ ID NO: 177) | 192 |
| | 7982_1R | TGGGTTTTCTTATGAAGGGG(SEQ ID NO: 178) | |
| 94 | 80548_1F | AAACACCCGTGGGCATATTA(SEQ ID NO: 179) | 228 |
| | 80548_1R | AAGCCTTGATTTTCTCTTCCAA(SEQ ID NO: 180) | |
| 95 | 86059_1F | CTGACATGTGTGAGTGTGAGAGAG(SEQ ID NO: 181) | 161 |
| | 86059_1R | CGCTTCTAGTTTCGTCCCTG(SEQ ID NO: 182) | |
| 96 | 900_1F | AATTTCCAAAACTGCGTTGA(SEQ ID NO: 183) | 182 |
| | 900_1R | GGGAGGGGTTTCACATTCTT(SEQ ID NO: 184) | |
| 97 | 9129_1F | AGTCTTCAGTTGCGTGAAAAA(SEQ ID NO: 185) | 276 |
| | 9129_1R | GGGGTAATTGTGTCCTATCCA(SEQ ID NO: 186) | |
| 98 | 9139_1F | AAAAATTCGGATACCCCCAC(SEQ ID NO: 187) | 225 |
| | 9139_1R | GAAGCAGAGAAGGAAAGAAGAGAA(SEQ ID NO: 188) | |
| 99 | 97731_1F | CCGTGGGCGTCTTATTTTT(SEQ ID NO: 189) | 227 |
| | 97731_1R | TTCCAATTATCTTCTCTCTCACATTG(SEQ ID NO: 190) | |
| 100 | 1859_1F | CGTTGTTGCTGTGGATCTGT(SEQ ID NO: 191) | 255 |
| | 1859_1R | AATTCGTCGATCAAACACCC(SEQ ID NO: 192) | |

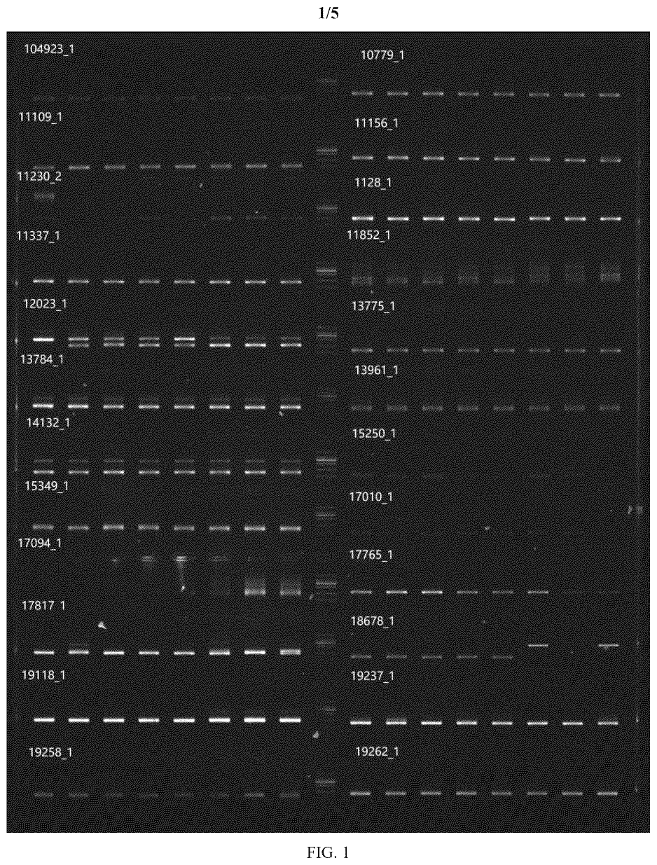

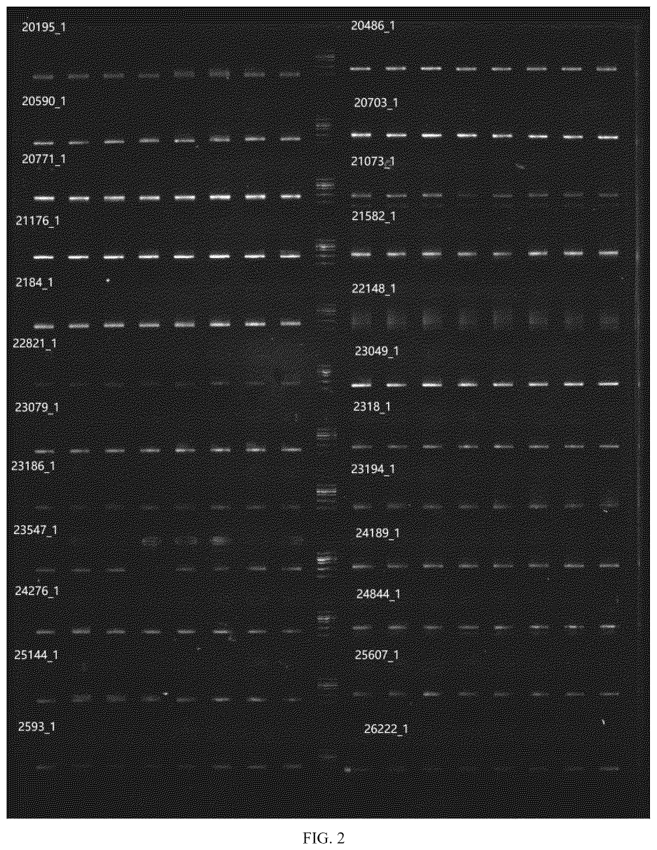

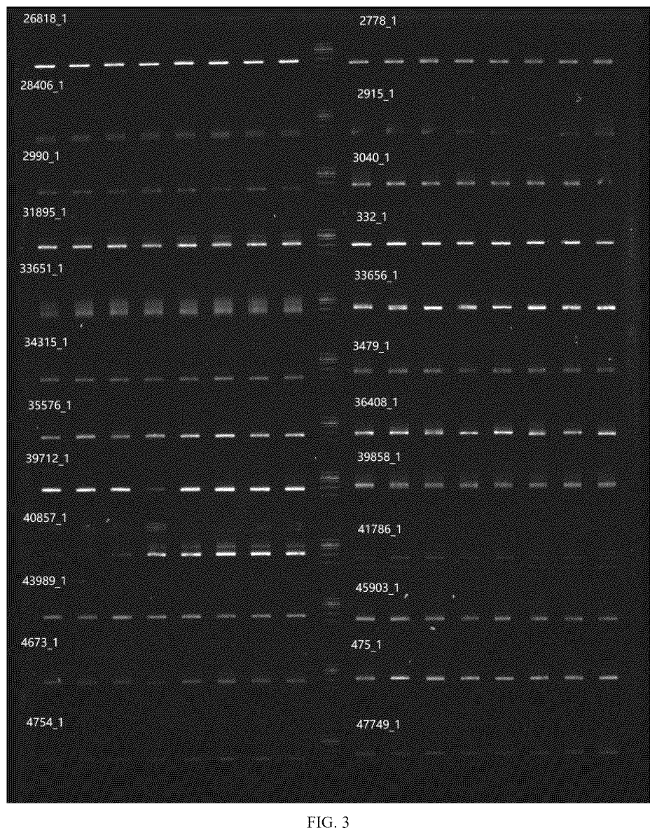

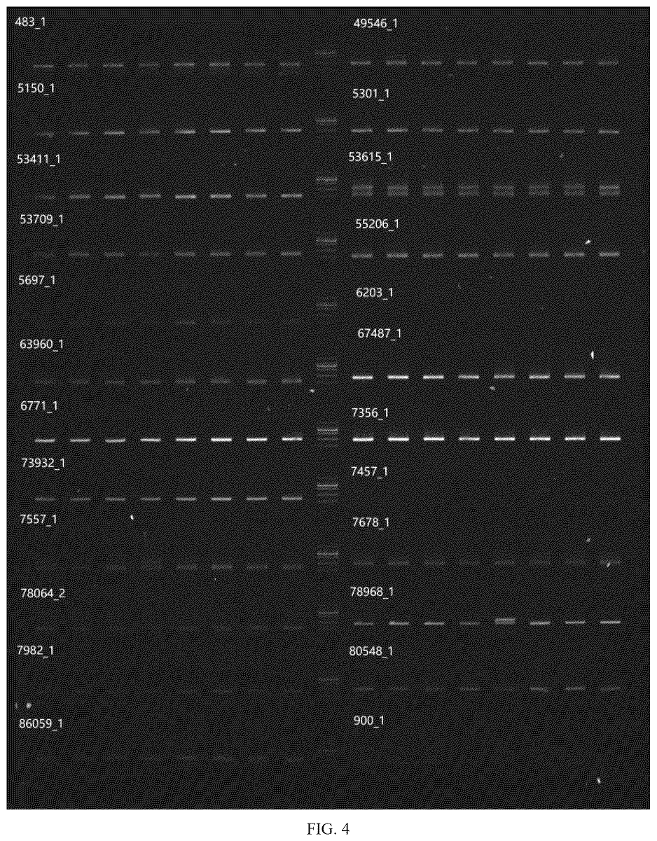

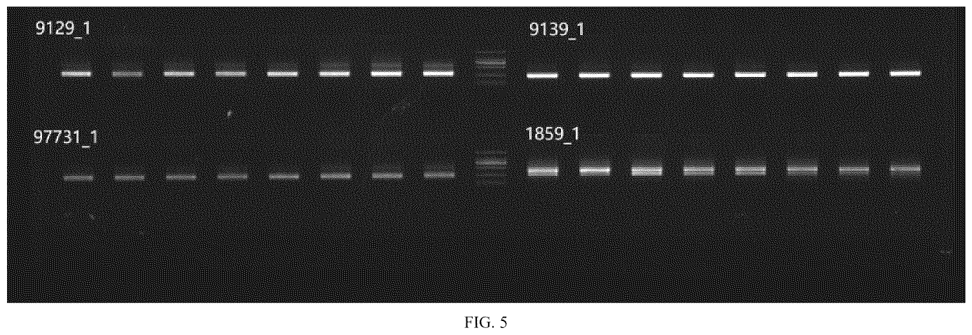

SSR primer screening: DNA was extracted from the leaves of 5 Astragalus membranaceus (Fisch.) Bge. var. mongholicus (Bge.) Hsiao resources and 4 Astragalus membranaceus (Fisch.) Bge. with large phenotypic differences from different sources (as shown in Table 2). The extracted DNA was amplified by PCR using the SSR primers designed in step (3). Polymorphism detection was then performed using 6-8% polyacrylamide gel electrophoresis to screen effective SSR primers. The results are shown in FIGS. 1 to 5. Among the 100 primer pairs listed, only the primer set of 18678_1F and 18678_1R provided by the present invention can clearly distinguish Astragalus membranaceus (Fisch.) Bge. var. mongholicus (Bge.) Hsiao and Astragalus membranaceus (Fisch.) Bge.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A primer set for identifying Astragalus membranaceus (Fisch.) Bge. var. mongholicus (Bge.) Hsiao, comprising a primer 18678_1F and a primer 18678_1R, wherein the primer 18678_1F has the nucleotide sequence set forth in SEQ ID NO: 1, and the primer 18678_1R has the nucleotide sequence set forth in SEQ ID NO: 2.

2. A kit for identifying the Astragalus membranaceus (Fisch.) Bge. var. mongholicus (Bge.) Hsiao, comprising the primer set according to claim 1 and a detection reagent.

3. The kit according to claim 2, wherein the detection reagent is a polymerase chain reaction (PCR) amplification reagent.

4. A method for identifying Astragalus membranaceus (Fisch.) Bge. var. mongholicus (Bge.) Hsiao by using the primer set according to claim 1, comprising the following steps:
1) extracting a genomic DNA from a to-be-detected sample;
2) performing a PCR amplification by using the genomic DNA obtained in the step 1) as a template and using the primer set to obtain an amplification product; and
3) detecting the amplification product obtained in the step 2), and if the amplification product has a single 144 bp sequence, determining that the to-be-detected sample is the Astragalus membranaceus (Fisch.) Bge. var. mongholicus (Bge.) Hsiao.

5. The method according to claim 4, wherein a material for extracting the genomic DNA from the to-be-detected sample in the step 1) is a seedling leaf.

6. The method according to claim 4, wherein the template in the step 2) has a concentration of 10-20 ng/μL.

7. The method according to claim 4, wherein the primer set in the step 2) has a concentration of 8-12 μM.

8. The method according to claim 6, wherein a PCR amplification system in the step 2), with a total volume of 20 μL, comprises the following components: 1 μL of the template, 0.5 μL of the primer 18678_1F, 0.5 μL of the primer 18678_1R, 10 μL of 2×MIX, and 8 μL of ddH₂O;
a program of the PCR amplification in the step 2) is as follows: a pre-denaturation at 95° C. for 5 min; 15 cycles of a denaturation at 95° C. for 30 s, an annealing at 60° C. for 30 s, and an extension at 72° C. for 30 s; 20 cycles of the pre-denaturation at 95° C. for 30 s, the annealing at 50° C. for 30 s, and the extension at 72° C. for 30 s; and finally the extension at 72° C. for 7 min.

9. The method according to claim 4, wherein a method for detecting the amplification product comprises a polyacrylamide gel electrophoresis or a fluorescent labeling capillary electrophoresis.

10. A method for Radix astragali-assisted breeding, comprising using the primer set according to claim 1.

11. The method according to claim 7, wherein a PCR amplification system in the step 2), with a total volume of 20 μL, comprises the following components: 1 μL of the template, 0.5 μL of the primer 18678_1F, 0.5 μL of the primer 18678_1R, 10 μL of 2×MIX, and 8 μL of ddH₂O;
a program of the PCR amplification in the step 2) is as follows: a pre-denaturation at 95° C. for 5 min; 15 cycles of a denaturation at 95° C. for 30 s, an annealing at 60° C. for 30 s, and an extension at 72° C. for 30 s; 20 cycles of the pre-denaturation at 95° C. for 30 s, the annealing at 50° C. for 30 s, and the extension at 72° C. for 30 s; and finally the extension at 72° C. for 7 min.

12. A method for Radix astragali-assisted breeding, comprising using the kit according to claim 2.


SEQUENCE LISTING (192 sequences)

SEQ ID NO: 1 - DNA, 20 bp, synthetic construct
  source 1..20: mol_type = other DNA
aaacccaaacgaagcacaac

SEQ ID NO: 2 - DNA, 20 bp, synthetic construct
  source 1..20: mol_type = other DNA
gaaaacaaaatccgtcccaa

SEQ ID NO: 3 - DNA, 23 bp, synthetic construct
  source 1..23: mol_type = other DNA
aaaatggaagagtgtggaagaaa

SEQ ID NO: 4 - DNA, 20 bp, synthetic construct
  source 1..20: mol_type = other DNA
tctcctctcctcacattcgc

SEQ ID NO: 5 - DNA, 20 bp, synthetic construct
  source 1..20: mol_type = other DNA
acattatttcccatgcccaa

SEQ ID NO: 6 - DNA, 22 bp, synthetic construct
  source 1..22: mol_type = other DNA
caaaatggaagagtgtggaaga

SEQ ID NO: 7 - DNA, 21 bp, synthetic construct
  source 1..21: mol_type = other DNA
cttgatgttcttggttgctca

SEQ ID NO: 8 - DNA, 20 bp, synthetic construct
  source 1..20: mol_type = other DNA
cacacataggtcgcatcacc

SEQ ID NO: 9 - DNA, 20 bp, synthetic construct
  source 1..20: mol_type = other DNA
atggttgagctccaaaatcg

SEQ ID NO: 10 - DNA, 20 bp, synthetic construct
  source 1..20: mol_type = other DNA
accctcagctgaaaaacacc

SEQ ID NO: 11 - DNA, 20 bp, synthetic construct
  source 1..20: mol_type = other DNA
aacttaggggtgatgggagg

SEQ ID NO: 12 - DNA, 20 bp, synthetic construct
  source 1..20: mol_type = other DNA
atcgttcaccggacgataag

SEQ ID NO: 13 - DNA, 20 bp, synthetic construct
  source 1..20: mol_type = other DNA
aaaccagctggaccaaacac

SEQ ID NO: 14 - DNA, 20 bp, synthetic construct
  source 1..20: mol_type = other DNA
aaagggttgctgggagtttt

SEQ ID NO: 15 - DNA, 20 bp, synthetic construct
  source 1..20: mol_type = other DNA
aaggcaaagcacacaaaggt

SEQ ID NO: 16 - DNA, 20 bp, synthetic construct
  source 1..20: mol_type = other DNA
ggtcccctcgttcttttctt

SEQ ID NO: 17 - DNA, 22 bp, synthetic construct
  source 1..22: mol_type = other DNA
aacgctttcttttatttctcgc

SEQ ID NO: 18 - DNA, 21 bp, synthetic construct
  source 1..21: mol_type = other DNA
gcggaattgtgaatgacaaga

SEQ ID NO: 19 - DNA, 20 bp, synthetic construct
  source 1..20: mol_type = other DNA
accacagaaccagaaatgcc

SEQ ID NO: 20 - DNA, 20 bp, synthetic construct
  source 1..20: mol_type = other DNA
ccagcaaccaaaaactcaga

SEQ ID NO: 21 - DNA, 20 bp, synthetic construct
  source 1..20: mol_type = other DNA
agcggacctgtccaatacag

SEQ ID NO: 22 - DNA, 21 bp, synthetic construct
  source 1..21: mol_type = other DNA
tttttgaatcacccatgaacc

SEQ ID NO: 23 - DNA, 20 bp, synthetic construct
  source 1..20: mol_type = other DNA
caggctcacgacgttaaaca

SEQ ID NO: 24 - DNA, 20 bp, synthetic construct
  source 1..20: mol_type = other DNA
aaactctcctcccccacact

SEQ ID NO: 25 - DNA, 23 bp, synthetic construct
  source 1..23: mol_type = other DNA
actccttgattttctcttccact

SEQ ID NO: 26 - DNA, 21 bp, synthetic construct
  source 1..21: mol_type = other DNA
tttaggcttcttttgcgtgaa

SEQ ID NO: 27 - DNA, 20 bp, synthetic construct
  source 1..20: mol_type = other DNA
ccacagggagagcctaagtg

SEQ ID NO: 28 - DNA, 27 bp, synthetic construct
  source 1..27: mol_type = other DNA
aaaattatttccaaaaatacagtgaga

SEQ ID NO: 29 - DNA, 24 bp, synthetic construct
  source 1..24: mol_type = other DNA
ttcattcttaaaagcgtctgtgtc

SEQ ID NO: 30 - DNA, 23 bp, synthetic construct
  source 1..23: mol_type = other DNA
ccagatctgttcaacaacattca

SEQ ID NO: 31 - DNA, 20 bp, synthetic construct
  source 1..20: mol_type = other DNA
ggcaaattgagaaacccaga

SEQ ID NO: 32 - DNA, 21 bp, synthetic construct
  source 1..21: mol_type = other DNA
aaacgattgaaacgaaacaaa

SEQ ID NO: 33 - DNA, 20 bp, synthetic construct
  source 1..20: mol_type = other DNA
ccgaaactgatcgctctctc

SEQ ID NO: 34 - DNA, 20 bp, synthetic construct
  source 1..20: mol_type = other DNA
cacccgtgagcgtcttattt

SEQ ID NO: 35 - DNA, 25 bp, synthetic construct
  source 1..25: mol_type = other DNA
ccactatcttctctctcacaattcc

SEQ ID NO: 36 - DNA, 20 bp, synthetic construct
  source 1..20: mol_type = other DNA
acccaggttggtaagctcct

SEQ ID NO: 37 - DNA, 20 bp, synthetic construct
  source 1..20: mol_type = other DNA
aagacgcattcaatcggaac

SEQ ID NO: 38 - DNA, 20 bp, synthetic construct
  source 1..20: mol_type = other DNA
cattcaatccgcctcaagat

SEQ ID NO: 39 - DNA, 25 bp, synthetic construct
  source 1..25: mol_type = other DNA
ggagaaagaagaaaagaaaagaagg

SEQ ID NO: 40 - DNA, 20 bp, synthetic construct
  source 1..20: mol_type = other DNA
acaaatttggccgctacaac

SEQ ID NO: 41 - DNA, 24 bp, synthetic construct
  source 1..24: mol_type = other DNA
aagacgagtttctgtaaaattggg

SEQ ID NO: 42 - DNA, 25 bp, synthetic construct
  source 1..25: mol_type = other DNA
aactagcaacccatagtgataaaaa

SEQ ID NO: 43 - DNA, 20 bp, synthetic construct
  source 1..20: mol_type = other DNA
ggaagaggctcctctggtct

SEQ ID NO: 44 - DNA, 26 bp, synthetic construct
  source 1..26: mol_type = other DNA
ccctattttgacattaaagttgtgaa

SEQ ID NO: 45 - DNA, 20 bp, synthetic construct
  source 1..20: mol_type = other DNA
gaagaaggcaatgcacacaa

SEQ ID NO: 46 - DNA, 20 bp, synthetic construct
  source 1..20: mol_type = other DNA
cacacacgcacacacttgag

SEQ ID NO: 47 - DNA, 20 bp, synthetic construct
  source 1..20: mol_type = other DNA
tggagggtgtcgaagtttct

SEQ ID NO: 48 - DNA, 20 bp, synthetic construct
  source 1..20: mol_type = other DNA
agagcacgcgttttcagttt

SEQ ID NO: 49 - DNA, 21 bp, synthetic construct
  source 1..21: mol_type = other DNA
aaatccctcgctaaccaaaaa

SEQ ID NO: 50 - DNA, 19 bp, synthetic construct
  source 1..19: mol_type = other DNA
gtgaagttcgcgagtgagg

SEQ ID NO: 51 - DNA, 20 bp, synthetic construct
  source 1..20: mol_type = other DNA
aatcgcatctcacttttggg

SEQ ID NO: 52 - DNA, 20 bp, synthetic construct
  source 1..20: mol_type = other DNA
cgaagtagatgcagagcgag

SEQ ID NO: 53 - DNA, 20 bp, synthetic construct
  source 1..20: mol_type = other DNA
cgtagaggaccaaaccgaaa

SEQ ID NO: 54 - DNA, 21 bp, synthetic construct
  source 1..21: mol_type = other DNA
aacagaaaaacgaaagagcga

SEQ ID NO: 55 - DNA, 20 bp, synthetic construct
  source 1..20: mol_type = other DNA
aaagaaagaaaggcatcgca

SEQ ID NO: 56 - DNA, 20 bp, synthetic construct
  source 1..20: mol_type = other DNA
cctctacgaaagcaccgaag

SEQ ID NO: 57 - DNA, 20 bp, synthetic construct
  source 1..20: mol_type = other DNA
ccatctgcctcaccattctt

SEQ ID NO: 58 - DNA, 22 bp, synthetic construct
  source 1..22: mol_type = other DNA
gaaggaagaggaagtggagaaa

SEQ ID NO: 59 - DNA, 21 bp, synthetic construct
  source 1..21: mol_type = other DNA
cacatgtgtgaggacgagaga

SEQ ID NO: 60 - DNA, 20 bp, synthetic construct
  source 1..20: mol_type = other DNA
atgcaaaacaagggttggag

SEQ ID NO: 61 - DNA, 20 bp, synthetic construct
  source 1..20: mol_type = other DNA
atgttgccggtttgattcat

SEQ ID NO: 62 - DNA, 20 bp, synthetic construct
  source 1..20: mol_type = other DNA
caaccaacaaccaacaacca

SEQ ID NO: 63 - DNA, 20 bp, synthetic construct
  source 1..20: mol_type = other DNA
agacagtgcgaaggaggaaa

SEQ ID NO: 64 - DNA, 20 bp, synthetic construct
  source 1..20: mol_type = other DNA
cacccgtcatgtttgatttg

SEQ ID NO: 65 - DNA, 20 bp, synthetic construct
  source 1..20: mol_type = other DNA
aaagaaacgagtggtggtgg

SEQ ID NO: 66 - DNA, 20 bp, synthetic construct
  source 1..20: mol_type = other DNA
ctcctcacactcttctccca

SEQ ID NO: 67 - DNA, 23 bp, synthetic construct
  source 1..23: mol_type = other DNA
tcattgttaaaagcgtctgtgtc

SEQ ID NO: 68 - DNA, 27 bp, synthetic construct
  source 1..27: mol_type = other DNA
ttcatcaagactgttataattggtaaa

SEQ ID NO: 69 - DNA, 20 bp, synthetic construct
  source 1..20: mol_type = other DNA
aagggagagagaggaggtcg

SEQ ID NO: 70 - DNA, 20 bp, synthetic construct
  source 1..20: mol_type = other DNA
gtggaaagaggttggagggt

SEQ ID NO: 71 - DNA, 20 bp, synthetic construct
  source 1..20: mol_type = other DNA
atgaaccccacccttacctt

SEQ ID NO: 72 - DNA, 20 bp, synthetic construct
  source 1..20: mol_type = other DNA
agaaggcgtgaatccacaag

SEQ ID NO: 73 - DNA, 20 bp, synthetic construct
  source 1..20: mol_type = other DNA
acagaccccattcaccaaaa

SEQ ID NO: 74 - DNA, 20 bp, synthetic construct
  source 1..20: mol_type = other DNA
tgtttggtttggaaatcgct

SEQ ID NO: 75 - DNA, 20 bp, synthetic construct
  source 1..20: mol_type = other DNA
aggaaaaccgaagggagaaa

SEQ ID NO: 76 - DNA, 21 bp, synthetic construct
  source 1..21: mol_type = other DNA
catgcaatgacaatggagatg

SEQ ID NO: 77 - DNA, 20 bp, synthetic construct
  source 1..20: mol_type = other DNA
ccgctttcttttatttcacg

SEQ ID NO: 78 - DNA, 20 bp, synthetic construct
  source 1..20: mol_type = other DNA
gaaagcggtaaattgcatga

SEQ ID NO: 79 - DNA, 26 bp, synthetic construct
  source 1..26: mol_type = other DNA
aaattttgactaaaaagggaaataca

SEQ ID NO: 80 - DNA, 26 bp, synthetic construct
  source 1..26: mol_type = other DNA
tttcattgttaaaagtgtgtgtgtct

SEQ ID NO: 81 - DNA, 21 bp, synthetic construct
  source 1..21: mol_type = other DNA
ggggtaattgtgtcacatcca

SEQ ID NO: 82 - DNA, 20 bp, synthetic construct
  source 1..20: mol_type = other DNA
ttaggcttcttttgcgtgaa

SEQ ID NO: 83 - DNA, 20 bp, synthetic construct
  source 1..20: mol_type = other DNA
attcccattatttcccaggc

SEQ ID NO: 84 - DNA, 20 bp, synthetic construct
  source 1..20: mol_type = other DNA
cgcccgatctgtaacgtact

SEQ ID NO: 85 - DNA, 22 bp, synthetic construct
  source 1..22: mol_type = other DNA
ggagtgttaatactgcaacgca

SEQ ID NO: 86 - DNA, 20 bp, synthetic construct
  source 1..20: mol_type = other DNA
atcagtagcggcaagcaaac

SEQ ID NO: 87 - DNA, 20 bp, synthetic construct
  source 1..20: mol_type = other DNA
aagtcaaaacctgctgggtg

SEQ ID NO: 88 - DNA, 20 bp, synthetic construct
  source 1..20: mol_type = other DNA
agtgatgcaatgtgcagagc

SEQ ID NO: 89 - DNA, 20 bp, synthetic construct
  source 1..20: mol_type = other DNA
cgttcatcgtcatcgtgatt

SEQ ID NO: 90 - DNA, 20 bp, synthetic construct
  source 1..20: mol_type = other DNA
aaaaatccaacggtctcgtg

SEQ ID NO: 91 - DNA, 20 bp, synthetic construct
  source 1..20: mol_type = other DNA
aaaaccaaacacccaaccac

SEQ ID NO: 92 - DNA, 22 bp, synthetic construct
  source 1..22: mol_type = other DNA
gaagggagagagagaaaattcg

SEQ ID NO: 93 - DNA, 20 bp, synthetic construct
  source 1..20: mol_type = other DNA
gtgcacattcacttccatgc

SEQ ID NO: 94 - DNA, 20 bp, synthetic construct
  source 1..20: mol_type = other DNA
cttggtgtgattggtgtgct

SEQ ID NO: 95 - DNA, 20 bp, synthetic construct
  source 1..20: mol_type = other DNA
gaccatgacaatgcatcagg

SEQ ID NO: 96 - DNA, 20 bp, synthetic construct
  source 1..20: mol_type = other DNA
aaagagcacggaatggaatg

SEQ ID NO: 97 - DNA, 20 bp, synthetic construct
  source 1..20: mol_type = other DNA
tgctccagctgagaagaaga

SEQ ID NO: 98 - DNA, 20 bp, synthetic construct
  source 1..20: mol_type = other DNA
accacaacactggacctcaa

SEQ ID NO: 99 - DNA, 23 bp, synthetic construct
  source 1..23: mol_type = other DNA
tcacatttcacaacaacactcaa

SEQ ID NO: 100 - DNA, 21 bp, synthetic construct
  source 1..21: mol_type = other DNA
ccgctttcttttatttctcgc

SEQ ID NO: 101 - DNA, 21 bp, synthetic construct
  source 1..21: mol_type = other DNA
cggtgacaaaaagttggtaaa

SEQ ID NO: 102 - DNA, 20 bp, synthetic construct
  source 1..20: mol_type = other DNA
ttggggatacgcttcaagtc

SEQ ID NO: 103 - DNA, 24 bp, synthetic construct
  source 1..24: mol_type = other DNA
aaaatcttctctctcacaattcca

SEQ ID NO: 104 - DNA, 20 bp, synthetic construct
  source 1..20: mol_type = other DNA
cgtgagtgcgtttttaaggc

SEQ ID NO: 105 - DNA, 20 bp, synthetic construct
  source 1..20: mol_type = other DNA
aggggtaattgtgtcgcatc

SEQ ID NO: 106 - DNA, 20 bp, synthetic construct
  source 1..20: mol_type = other DNA
atacgcgctgattaggatgc

SEQ ID NO: 107 - DNA, 24 bp, synthetic construct
  source 1..24: mol_type = other DNA
tcatgaccatattgagagatcaga

SEQ ID NO: 108 - DNA, 20 bp, synthetic construct
  source 1..20: mol_type = other DNA
aaaccttgtcgcagtatggg

SEQ ID NO: 109 - DNA, 20 bp, synthetic construct
  source 1..20: mol_type = other DNA
gacgacaacgagaacgacaa

SEQ ID NO: 110 - DNA, 20 bp, synthetic construct
  source 1..20: mol_type = other DNA
gattctgggatgtgtttggc

SEQ ID NO: 111 - DNA, 21 bp, synthetic construct
  source 1..21: mol_type = other DNA
ggtcagagagaaaagggatgg

SEQ ID NO: 112 - DNA, 25 bp, synthetic construct
  source 1..25: mol_type = other DNA
aaattgttatgtctgctaatgtgga

SEQ ID NO: 113 - DNA, 20 bp, synthetic construct
  source 1..20: mol_type = other DNA
agaccaatagggcacacagc

SEQ ID NO: 114 - DNA, 25 bp, synthetic construct
  source 1..25: mol_type = other DNA
tgcgtgaaaaataaagaaagatatg

SEQ ID NO: 115 - DNA, 21 bp, synthetic construct
  source 1..21: mol_type = other DNA
caactgggttgattttgattg

SEQ ID NO: 116 - DNA, 20 bp, synthetic construct
  source 1..20: mol_type = other DNA
cctgctgcaacagtgtgttc

SEQ ID NO: 117 - DNA, 20 bp, synthetic construct
  source 1..20: mol_type = other DNA
agaactcagcctcaccatgc

SEQ ID NO: 118 - DNA, 20 bp, synthetic construct
  source 1..20: mol_type = other DNA
acgactaacccatgccaatc

SEQ ID NO: 119 - DNA, 20 bp, synthetic construct
  source 1..20: mol_type = other DNA
aaggggaatgggaaagattg

SEQ ID NO: 120 - DNA, 20 bp, synthetic construct
  source 1..20: mol_type = other DNA
cactatggtcatttcgccct

SEQ ID NO: 121 - DNA, 19 bp, synthetic construct
  source 1..19: mol_type = other DNA
ctttgcgtgagtgcgtttt

SEQ ID NO: 122 - DNA, 22 bp, synthetic construct
  source 1..22: mol_type = other DNA
gggtatttgtgtcacatccctt

SEQ ID NO: 123 - DNA, 20 bp, synthetic construct
  source 1..20: mol_type = other DNA
actcttggagagccacctga

SEQ ID NO: 124 - DNA, 20 bp, synthetic construct
  source 1..20: mol_type = other DNA
aattctcctcccccacactt

SEQ ID NO: 125 - DNA, 20 bp, synthetic construct
  source 1..20: mol_type = other DNA
atgattttgcaatttaccgc

SEQ ID NO: 126 - DNA, 22 bp, synthetic construct
  source 1..22: mol_type = other DNA
aagcgagaaataaaagaaagcg

SEQ ID NO: 127 - DNA, 22 bp, synthetic construct
  source 1..22: mol_type = other DNA
caaaatggatgaataatgcgaa

SEQ ID NO: 128 - DNA, 20 bp, synthetic construct
  source 1..20: mol_type = other DNA
atggcgttgaatttgtaggc

SEQ ID NO: 129 - DNA, 23 bp, synthetic construct
  source 1..23: mol_type = other DNA
acgcaatttacaattctgtcatt

SEQ ID NO: 130 - DNA, 20 bp, synthetic construct
  source 1..20: mol_type = other DNA
cccaataacccgagactttg

SEQ ID NO: 131 - DNA, 20 bp, synthetic construct
  source 1..20: mol_type = other DNA
acccgtgggcttcttatttt

SEQ ID NO: 132 - DNA, 23 bp, synthetic construct
  source 1..23: mol_type = other DNA
actccttgactttctcttccact

SEQ ID NO: 133 - DNA, 20 bp, synthetic construct
  source 1..20: mol_type = other DNA
cccccggttaactcttcaat

SEQ ID NO: 134 - DNA, 21 bp, synthetic construct
  source 1..21: mol_type = other DNA
cgagattcaagaagaagggaa

SEQ ID NO: 135 - DNA, 26 bp, synthetic construct
  source 1..26: mol_type = other DNA
cccaagactttgtatctactcactca

SEQ ID NO: 136 - DNA, 24 bp, synthetic construct
  source 1..24: mol_type = other DNA
catcttcctcttcttcactctcag

SEQ ID NO: 137 - DNA, 20 bp, synthetic construct
  source 1..20: mol_type = other DNA
agatgatgatgcacaaccga

SEQ ID NO: 138 - DNA, 20 bp, synthetic construct
  source 1..20: mol_type = other DNA
aacacatcatggctccaaca

SEQ ID NO: 139 - DNA, 20 bp, synthetic construct
  source 1..20: mol_type = other DNA
gccgtgtgtggatgttgata

SEQ ID NO: 140 - DNA, 26 bp, synthetic construct
  source 1..26: mol_type = other DNA
caattttatcttacgttcatctgtca

SEQ ID NO: 141 - DNA, 20 bp, synthetic construct
  source 1..20: mol_type = other DNA
aaaggaagagcgtgcttcaa

SEQ ID NO: 142 - DNA, 20 bp, synthetic construct
  source 1..20: mol_type = other DNA
aaggaaagggctggtgaaat

SEQ ID NO: 143 - DNA, 25 bp, synthetic construct
  source 1..25: mol_type = other DNA
gatcaaaatatgaaatgtgatgaga

SEQ ID NO: 144 - DNA, 20 bp, synthetic construct
  source 1..20: mol_type = other DNA
cattgttaaaagcgcctgtg

SEQ ID NO: 145 - DNA, 26 bp, synthetic construct
  source 1..26: mol_type = other DNA
aattttgactaaaaagggaaatacat

SEQ ID NO: 146 - DNA, 20 bp, synthetic construct
  source 1..20: mol_type = other DNA
aactcaagagacgaccacgc

SEQ ID NO: 147 - DNA, 20 bp, synthetic construct
  source 1..20: mol_type = other DNA
gattgtcgggcagtgtatcc

SEQ ID NO: 148 - DNA, 21 bp, synthetic construct
  source 1..21: mol_type = other DNA
aaggaggaaattccattctgc

SEQ ID NO: 149 - DNA, 20 bp, synthetic construct
  source 1..20: mol_type = other DNA
gatgggcatccatcgtttac

SEQ ID NO: 150 - DNA, 20 bp, synthetic construct
  source 1..20: mol_type = other DNA
caggaggcaacctgtgtttt

SEQ ID NO: 151 - DNA, 24 bp, synthetic construct
  source 1..24: mol_type = other DNA
tgatcaaatagaaacaaaaggaaa

SEQ ID NO: 152 - DNA, 20 bp, synthetic construct
  source 1..20: mol_type = other DNA
acaatctcagcgatgcaatg

SEQ ID NO: 153 - DNA, 19 bp, synthetic construct
  source 1..19: mol_type = other DNA
actcacacacacgcacaca

SEQ ID NO: 154 - DNA, 20 bp, synthetic construct
  source 1..20: mol_type = other DNA
acctgtgggaaacaggtgag

SEQ ID NO: 155 - DNA, 20 bp, synthetic construct
  source 1..20: mol_type = other DNA
gattgacggccttgtgtttt

SEQ ID NO: 156 - DNA, 20 bp, synthetic construct
  source 1..20: mol_type = other DNA
aaaggagaacatgcagggtg

SEQ ID NO: 157 - DNA, 25 bp, synthetic construct
  source 1..25: mol_type = other DNA
cattgatgaaacagaattttcaaga

SEQ ID NO: 158 - DNA, 20 bp, synthetic construct
  source 1..20: mol_type = other DNA
accaccatctctgatgagcc

SEQ ID NO: 159 - DNA, 22 bp, synthetic construct
  source 1..22: mol_type = other DNA
ttggtgttcttgaggagagaga

SEQ ID NO: 160 - DNA, 20 bp, synthetic construct
  source 1..20: mol_type = other DNA
aacgaaatgcaaaagggaga

SEQ ID NO: 161 - DNA, 21 bp, synthetic construct
  source 1..21: mol_type = other DNA
cgagaagcagagaaggaaaga

SEQ ID NO: 162 - DNA, 20 bp, synthetic construct
  source 1..20: mol_type = other DNA
atcagcgcctatttcaacct

SEQ ID NO: 163 - DNA, 20 bp, synthetic construct
  source 1..20: mol_type = other DNA
ccgaccttcgcaacagtaat

SEQ ID NO: 164 - DNA, 20 bp, synthetic construct
  source 1..20: mol_type = other DNA
ccatgcccaaaaattcaaaa

SEQ ID NO: 165 - DNA, 20 bp, synthetic construct
  source 1..20: mol_type = other DNA
ccaaagagggaaatttgggt

SEQ ID NO: 166 - DNA, 20 bp, synthetic construct
  source 1..20: mol_type = other DNA
aatcgcaaaattggtgcttt

SEQ ID NO: 167 - DNA, 23 bp, synthetic construct
  source 1..23: mol_type = other DNA
caccaaattaaattcaccaaacc

SEQ ID NO: 168 - DNA, 23 bp, synthetic construct
  source 1..23: mol_type = other DNA
aaagaaaagaaagaaaggaagcg

SEQ ID NO: 169 - DNA, 20 bp, synthetic construct
  source 1..20: mol_type = other DNA
tctctccctttcacccagtc

SEQ ID NO: 170 - DNA, 21 bp, synthetic construct
  source 1..21: mol_type = other DNA
acgttcatctgtcacgttttt

SEQ ID NO: 171 - DNA, 20 bp, synthetic construct
  source 1..20: mol_type = other DNA
attttgcgtgagtgcgtttt

SEQ ID NO: 172 - DNA, 22 bp, synthetic construct
  source 1..22: mol_type = other DNA
ttgctccttgattttctcttcc

SEQ ID NO: 173 - DNA, 20 bp, synthetic construct
  source 1..20: mol_type = other DNA
acctttgtgtgcattgcctt

SEQ ID NO: 174 - DNA, 23 bp, synthetic construct
  source 1..23: mol_type = other DNA
tcgaaaatgaaagtgatgatgaa

SEQ ID NO: 175 - DNA, 20 bp, synthetic construct
  source 1..20: mol_type = other DNA
ccctcttctgccattggtaa

SEQ ID NO: 176 - DNA, 20 bp, synthetic construct
  source 1..20: mol_type = other DNA
aggacttaccctaaacccgc

SEQ ID NO: 177 - DNA, 20 bp, synthetic construct
  source 1..20: mol_type = other DNA
aagggggaagtgtcctcaat

SEQ ID NO: 178 - DNA, 20 bp, synthetic construct
  source 1..20: mol_type = other DNA
tgggttttcttatgaagggg

SEQ ID NO: 179 - DNA, 20 bp, synthetic construct
  source 1..20: mol_type = other DNA
aaacacccgtgggcatatta

SEQ ID NO: 180 - DNA, 22 bp, synthetic construct
  source 1..22: mol_type = other DNA
aagccttgattttctcttccaa

SEQ ID NO: 181 - DNA, 24 bp, synthetic construct
  source 1..24: mol_type = other DNA
ctgacatgtgtgagtgtgagagag

SEQ ID NO: 182 - DNA, 20 bp, synthetic construct
  source 1..20: mol_type = other DNA
cgcttctagtttcgtccctg

SEQ ID NO: 183 - DNA, 20 bp, synthetic construct
  source 1..20: mol_type = other DNA
aatttccaaaactgcgttga

SEQ ID NO: 184 - DNA, 20 bp, synthetic construct
  source 1..20: mol_type = other DNA
gggaggggtttcacattctt

SEQ ID NO: 185 - DNA, 21 bp, synthetic construct
  source 1..21: mol_type = other DNA
agtcttcagttgcgtgaaaaa

SEQ ID NO: 186 - DNA, 21 bp, synthetic construct
  source 1..21: mol_type = other DNA
ggggtaattgtgtcctatcca

SEQ ID NO: 187 - DNA, 20 bp, synthetic construct
  source 1..20: mol_type = other DNA
aaaaattcggatacccccac

SEQ ID NO: 188 - DNA, 24 bp, synthetic construct
  source 1..24: mol_type = other DNA
gaagcagagaaggaaagaagagaa

SEQ ID NO: 189 - DNA, 19 bp, synthetic construct
  source 1..19: mol_type = other DNA
ccgtgggcgtcttattttt

SEQ ID NO: 190 - DNA, 26 bp, synthetic construct
  source 1..26: mol_type = other DNA
ttccaattatcttctctctcacattg

SEQ ID NO: 191 - DNA, 20 bp, synthetic construct
  source 1..20: mol_type = other DNA
cgttgttgctgtggatctgt

SEQ ID NO: 192 - DNA, 20 bp, synthetic construct
  source 1..20: mol_type = other DNA
aattcgtcgatcaaacaccc